(19) United States
(12) Patent Application Publication
(10) Pub. No.: US 2008/0090258 A1
(43) Pub. Date: Apr. 17, 2008

(54) Method and composition for diagnosing endometrial cancer

(75) Inventor: Anna Lokshin, Pittsburgh, PA (US)

Correspondence Address: THE WEBB LAW FIRM, P.C., 700 KOPPERS BUILDING, 436 SEVENTH AVENUE, PITTSBURGH, PA, 15219, US

(73) Assignee: University of Pittsburgh -of the Commonwealth System of Higher Education, Pittsburgh, PA (US)

(21) Appl. No.: 11/823,848

(22) Filed: Jun. 28, 2007

Related U.S. Application Data
(60) Provisional application No. 60/817,064, filed on Jun. 28, 2006.

(51) Int. Cl. G01N 33/53

(52) U.S. Cl. 435/7.23

(57) ABSTRACT

The present invention is related to methods and reagents for a multifactorial assay for the rapid, early detection of cancer and, more particularly, is related to a multimarker serological diagnostic test for early detection of endometrial cancer.

### CROSS REFERENCE TO RELATED APPLICATIONS

This application claims priority to U.S. Provisional Patent Application No. 60/817,064, filed on Jun. 28, 2006, which is incorporated herein by reference.

### SUMMARY OF THE INVENTION

The present invention is related to methods and reagents for a multifactorial assay for the rapid, early detection of cancer and, more particularly, is related to a multimarker serological diagnostic test for early detection of endometrial cancer. The multimarker serological diagnostic test operates by determining the level of at least two markers. The markers are selected from the group consisting of CA 125, CA 15-3, CA 19-9, CEA, AFP, CA 72-4, VEGF, bFGF, IGFBPI, HGF, ErbB2, EGFR, TGFα, Fas, FasL, Cyfra 21-1, MMP-1, MMP-2, MMP-3, MMP-7, MMP-8, MMP-9, MMP-12, MMP-13, tPAI, sICAM, sVCAM, sE-selectin, adiponectin, resistin, IL-6, IL-8, TNFα, TNFR I, G-CSF, CD40L, IL-2R, IP-10, MCP-1, MIP-1α, MIP-1β, MIF, eotaxin, RANTES, FSH, LH, TSH, ACTH, prolactin, GH, βHCG, hK8, hK10, active PAI-1, ULBP-1, ULBP-2, ULBP-3, MICA, angiostatin, SCC, serum amyloid A (“SAA”), TTR, S100, mesothelin, and myeloperoxidase (MPO).

### DESCRIPTION OF THE PREFERRED EMBODIMENT(S)

One embodiment of the invention is a method of diagnosing endometrial cancer comprising determining the levels of at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or twenty-five of sixty-five blood markers selected from the group consisting of CA 125, CA 15-3, CA 19-9, CEA, AFP, CA 72-4, VEGF, bFGF, IGFBPI, HGF, ErbB2, EGFR, TGFα, Fas, FasL, Cyfra 21-1, MMP-1, MMP-2, MMP-3, MMP-7, MMP-8, MMP-9, MMP-12, MMP-13, tPAI, sICAM, sVCAM, sE-selectin, adiponectin, resistin, IL-6, IL-8, TNFα, TNFR I, G-CSF, CD40L, IL-2R, IP-10, MCP-1, MIP-1α, MIP-1β, MIF, eotaxin, RANTES, FSH, LH, TSH, ACTH, prolactin, GH, βHCG, hK8, hK10, active PAI-1, ULBP-1, ULBP-2, ULBP-3, MICA, angiostatin, SCC, serum amyloid A, TTR, S100, mesothelin, and myeloperoxidase (MPO). In a further embodiment of this embodiment of the invention, prolactin is one of the markers used to diagnose endometrial cancer.

Another embodiment of the invention is a method of diagnosing endometrial cancer comprising determining the levels of at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or twenty-five of forty-one blood markers selected from the group consisting of IL-6, MIP-1α, MIP-1β, TNFRI, IL-2R, IGFBP-I, TSH, prolactin, GH, ACTH, TGFβ, MMP-7, MICA, SCC, SAA, IL-8, eotaxin, VEGF, CA 19-9, CA 125, ErbB2, EGFR, AFP, mesothelin, FSH, LH, CD40L, sVCAM-1, sICAM-1, tPAI-1, MPO, adiponectin, MMP-2, MMP-3, MMP-8, MMP-9, ULBP-1, ILBP-3, TTR, sFas, and sFasL. In a further embodiment of this embodiment of the invention, prolactin is one of the markers used to diagnose endometrial cancer.

In another embodiment, the method of diagnosing endometrial cancer comprises determining the expression of at least two, three, four, five, six, seven, eight, nine, ten, eleven, twelve, thirteen, fourteen or fifteen of fifteen up-regulated markers selected from the group consisting of IL-6, MIP-1α, MIP-1β, TNFRI, IL-2R, IGFBP-I, TSH, prolactin, GH, ACTH, TGFβ, MMP-7, MICA, SCC, and SAA. In a further embodiment of this embodiment of the invention, prolactin is one of the markers used to diagnose endometrial cancer.

In another embodiment, the method of diagnosing endometrial cancer comprises determining the level of expression of at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four twenty-five or twenty-six of twenty-six down-regulated markers selected from the group consisting of IL-8, eotaxin, VEGF, CA 19-9, CA 125, ErbB2, EGFR, AFP, mesothelin, FSH, LH, CD40L, sVCAM-1, sICAM-1, tPAI-1, MPO, adiponectin, MMP-2, MMP-3, MMP-8, MMP-9, ULBP-1, ILBP-3, TTR, sFas, and sFasL.

In another embodiment, the method of diagnosing endometrial cancer comprises determining the level of at least one, two, three, four, five, six, seven, eight, nine, ten, eleven, twelve, thirteen, fourteen or fifteen up-regulated marker(s) listed above, and at least one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four twenty-five or twenty-six down-regulated marker(s) listed above. In a further embodiment of this embodiment of the invention, prolactin is one of the up-regulated markers used to diagnose endometrial cancer.

Another embodiment of the invention is a method of diagnosing endometrial cancer comprising determining the levels of at least two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or twenty five of thirty blood markers selected from the group consisting of CA 15-3, CA 19-9, CA 125, CEA, CA 72-4, AFP, ErbB2, EGFR, kallikrein 6, kallikrein 8, kallikrein 10, Fas, FasL, Cyfra 21-1, TPA/TPS, IGFBP1, S100, angiostatin, SSC, ULBP1, ULBP 2, ULBP 3, βHCG, MICA, HE4, SMRP, mesothelin, SAA, and TTR. In a further embodiment of this embodiment of the invention, prolactin is one of the markers used to diagnose endometrial cancer.

Another embodiment of the invention is a method of determining endometrial cancer comprising determining the levels of at least two, three, four, five, six, seven, eight, nine, ten, eleven, twelve, thirteen, fourteen, fifteen, sixteen, seventeen, eighteen, nineteen, twenty of twenty blood markers selected from the group consisting of prolactin, TTR, CA 19-9, ULBP-2, CD40L, MMP-9, FSH, adiponectin, TSH, ACTH, eotaxin, TNFR-I, EGFR, IL-8, S-100, AFP, mesothelin, MMP-3, CEA, IGFBP-I. In a further embodiment of this embodiment of the invention, prolactin is one of the markers used to diagnose endometrial cancer.

Another embodiment of the invention is a method of determining endometrial cancer comprising determining the levels of prolactin, GH, eotaxin, e-selectin and FSH. In this embodiment, a positive diagnosis of endometrial cancer is made if it is determined that prolactin and GH are up-regulated, and that eotaxin, e-selectin and FSH are down-regulated.

The procedure used to develop the new method was as follows. Capture antibodies were monoclonal, and detection antibodies were polyclonal. Capture antibodies were biotinylated using EZ-Link Sulfo-NHS-Biotinylation Kit (Pierce, Rockford, Ill.) according to the manufacturer's protocol. The extent of biotin incorporation was determined using the HABA assay and was typically ˜20 moles of biotin per mole of protein. Capture antibody was covalently coupled to carboxylated polystyrene microspheres number 74 purchased from Luminex Corporation (Austin, Tex.). Covalent coupling of the capture antibodies to the microspheres was performed following the procedures recommended by Luminex. Coupling efficiency of the monoclonal antibodies was tested by staining 2000 microspheres with PE-conjugated goat anti-mouse IgG (BD Biosciences, San Diego, Calif.). The assay was further optimized for concentration of the detection antibody and for incubation times. The results of analytical assay validation are presented in Table 3 for representative assays. Intra-assay variability, expressed as a coefficient of variation, was calculated based on the average for 10 patient samples and measured twice at two different time points. Every individual Luminex assay that was used in our study has been validated according to commercial standards for: a.) sensitivity, b). inter- and intra-assay reproducibility, c.) % recovery from serum, and d.) against conventional single analyte ELISA. Additionally, performance of each assay singly is compared to that when multiplexed to make sure that there is no cross-reactivity. The results of assay validation shown in Table 1 confirm that each individual assay demonstrated high sensitivity and reproducibility, as well as recovery and correlation with appropriate ELISA. Finally, these microspheres were combined into a multiplex panel and the performance of each marker in the multiplexed panel was re-validated versus the performance of the same marker in single-plex format. Overall, including commercially available multiplexed bead-based assays, we possess assays for analysis of 93 cancer-related biomarkers (Table 2).

TABLE 1
Validation of multiplexed xMAP ® assays developed in UPCI Luminex
Core Facility

| Biomarkers | CA19-9 | CA125 | CEA | CA15-3 | ErbB2 | EGFR | Fas | FasL | Cyfra21 |
|---|---|---|---|---|---|---|---|---|---|
| Sensitivity | 20 IU/ml | 5 U/ml | 50 U/ml | 1 IU/ml | 50 pg/ml | 5 pg/ml | 15 pg/ml | 50 pg/ml | 300 pg/ml |
| Recovery (serum), % | 96 | 101 | 99 | 98 | 100 | 98 | 98 | 95 | 89 |
| Intra-assay | 8.6 | 6.0 | 8.2 | 7.9 | 6.5 | 7.1 | 5.5 | 6.2 | 5.1 |
| variation, % CV | | | | | | | | | |
| Inter-assay | 5.4 | 6.2 | 6.4 | 5.1 | 7.1 | 6.8 | 6.1 | 3.2 | 5.3 |
| variation (% CV) | | | | | | | | | |
| Correlation with | 0.97 | 0.98 | 0.98 | 0.97 | 0.99 | 0.98 | 0.99 | 0.99 | 0.98 |
| ELISA | | | | | | | | | |

TABLE 2
Available multiplexed biomarkers

| Biological groups | Proteins |
|---|---|
| Cytokines | IL-1β, IL-2, IL-4, IL-5, IL-6, IL-8, IL-10, |
| | IL-12p40, IL-13, IL-15, IL-17, IL-18, TNFα, |
| | TNFR I, TNFR II, IFNγ, GM-CSF, G-CSF, |
| | IL-2R, IL-6R, IL-1α, IFNα, IL-1Rα |
| Chemokines | RANTES, MIP-1α, MIP-1β, MCP-1,2,3, Eotaxin, |
| | MIG, MIF, IP-10 |
| Growth/angiogenic | EGF, EGFR, VEGF, bFGF, HGF, NGF, Her2/neu, |
| factors | IGFBPI |
| Cancer Antigens | CA 125, CA 15-3, CEA, AFP, CA 19-9, CA 72-4, |
| | PSA |
| Apoptotic proteins | Cyfra 21-1, TPA, sDR5, sFas, sFasL |
| Proteases | Kallikreins 6, 8, 10, 11; MMP-1,2,3,7,9 |
| Adhesion molecules | sICAM, sVCAM, sE-selectin |
| Other markers | mesothelin, SMR, HE4, tPAI-1, active PAI-1, |
| | ULBP-1, ULBP-2, ULBP-3, MICA, angiostatin, |
| | SCC, serum amyloid A, transthyretin, |
| | apolipoprotein H, S100, MPO, Hsp27 |
| Hormones | prolactin, TSH, βHCG, LH, ACTH, GH |
| Adipokines | adiponectin, leptin, resistin |

Multiplex analysis of serum concentrations of different biomarkers in endometrial cancer patients.

A preliminary bead-based 65-biomarker xMAP® panel, including most potential endometrial cancer serum biomarkers, was utilized to screen sera of 115 patients with endometrial cancer and 135 age-matched healthy controls (Table 3). The biomarkers included cancer antigens, CA 125, CA 15-3, CA 19-9, CEA, AFP, CA 72-4, VEGF, bFGF, IGFBPI, HGF, ErbB2, EGFR, TGFα, Fas, FasL, Cyfra 21-1, MMP-1, MMP-2, MMP-3, MMP-7, MMP-8, MMP-9, MMP-12, MMP-13, tPAI, sICAM, sVCAM, sE-selectin, adiponectin, resistin, IL-6, IL-8, TNFα, TNFR I, G-CSF, CD40L, IL-2R, IP-10, MCP-1, MIP-1α, MIP-1β, MIF, eotaxin, RANTES, FSH, LH, TSH, ACTH, Prolactin, GH, βHCG, hK8, hK10, active PAI-1, ULBP-1, ULBP-2, ULBP-3, MICA, angiostatin, SCC, serum amyloid A, TTR, S100, mesothelin, and myeloperoxidase (MPO).

TABLE 3
Patient Characteristics

| Patient Group | Age | | Tumor Grade | Cancer Stage |
|---|---|---|---|---|
| Control | N = 135 | | | |
| | Range | 22-84 | | |
| | Median | 57 | | |
| | Average | 60.2 | | |
| Endometrial | N = 115 | | 1-3 | 1-6 |
| Cancer | Range | 25-92 | | |
| | Median | 58.5 | | |
| | Average | 62.4 | | |

Serum concentrations of IL-6, MIP-1α, MIP-1β, TNFRI, IL-2R, IGFBP-I, TSH, Prolactin, GH, ACTH, TGFβ, MMP-7, MICA, SCC, and SAA were significantly elevated in patients with endometrial cancer as compared to healthy controls. In contrast, serum levels of IL-8, Eotaxin, VEGF, CA 19-9, CA 125, ErbB2, EGFR, AFP, Mesothelin, FSH, LH, CD40L, sVCAM-1, sICAM-1, tPAI-1, MPO, adiponectin, MMP-2,3,8,9, ULBP-1,3, TTR, sFas, and sFasL were significantly lower in patients with endometrial cancer compared to healthy individuals. Serum levels of other proteins were not statistically different in tested clinical groups. Prolactin was the strongest discriminative biomarker for endometrial cancer providing alone 95% sensitivity at 98% specificity. None of the other biomarkers was able to provide classification accuracy higher than 40% sensitivity at 50% specificity. We have performed multivariate analysis to identify a combination of biomarkers that would further improve diagnostic power offered by prolactin.

Development of adaptive density estimator—Projection Pursuit (ADEPT) algorithm.

A novel approach to the multivariate two-class events classification of sparse data in a multidimensional space has been developed based on classification in multiple k-dimensional projections with subsequent applying a decision fusion algorithm to form a final classifier. The discrimination within a single k-dimensional projection was performed using a kernel based probability density estimator with adoptive bandwidth (ADE) by creating a separate density probability estimations for both classification events and then generating the logit score reflecting the probability of a given data point to fall into one of the two event classes. The resulting score was obtained as a weighted sum of scores over all selected projections. The optimal set of projections was then obtained by utilizing the projection pursuit technique (PT) applied to the simulation set which was comprised of series of the training subsets created by repetitive random sub-sampling of the original data set and adding the Gaussian white noise to each data point as well as a scale noise to the whole training subset in the form of the linear transform with random coefficients in order to reflect the scale de-synchronization between successive multiplexed runs. The projections were chosen in such a way that when combined together they maximized the discrimination rate simultaneously for all training subsets.

Modified Fisher Discrimination Algorithm (MFD).

Alternatively, Modified Fisher Discriminant (M-FDA) analysis was used as follows. The optimal scoring function (SF) for a given set of markers was constructed as a linear combination of logarithm concentrations measured for each marker in the set. The optimal SF is deduced from data in the training database containing two identified groups of cases. The dimensionality of the solution vector (coefficients in the linear combination representing SF) is equal to the number of markers in the set, N. Thus, the score S for each data is a scalar product of solution vector and data vector (the set of N TFI measured for each case). The optimal SF is found by the following procedure. First, the solution vector with fixed length that minimizes the functional

$$Ω = \frac{\overline{Δ {S}_{1}^{2}} + \overline{Δ {S}_{1}^{2}}}{{( \overline{{S}_{1}} - \overline{{S}_{2}} )}^{2}} .$$

is found. Here S̅₁̅ and S̅₂̅ are the average scores for both groups in the training set while Δ̅S̅₁̅²̅ and Δ̅S̅₂̅²̅ are the mean squared deviations of the score for each case from the average score in its group. The solution is then refined by the procedure in which each case is assigned a weight based on its score. The closer score of the case to the average score of the opposite group, the higher weight is given to the case. In this way the discrimination between the close score cases from different groups is improved at the expense of some reduction in the scoring differential of well discriminated cases. The refining procedure is iterated until the best overall discrimination between two groups is achieved. With limited statistics in the training set and large number of markers the mathematical problem of minimization of functional Ω is ill-conditioned. That means that the optimal SF is unstable and most probably reflects the random noise in data within the training set rather then the actual diagnostic information.

Preliminary mathematical analysis indicates that to be stable against random fluctuation in the training set, SF should be based on no more then 8-9 markers. To find the subset of markers that gives the best stable SF the following procedure is used. First, all combinations of 6 markers are used to generate optimal SF according to procedure above, and one thousand combinations that provide best discrimination between training groups are retained. With 45 markers total, there are about 10⁷ different combinations of 6 markers to evaluate. Then 15 markers that are most common within these best sets of six markers are selected. The best subsets of markers from this reduced set of 15 are then selected by reviewing all possible combinations. The 100 subsets of each size are retained.

These subsets are subjected to cross-validation in the following way. Each case in the training group is randomly excluded from determination of optimal SF with probability of 10%. The excluded cases are then diagnosed and the percentage of misdiagnosed cases calculated. This cross-validation procedure is repeated until the probability of incorrect diagnosis for each subset of markers is known with the accuracy of few percent.

Multimarker approach for early detection of endometrial cancer.

The data for 65 markers were then analyzed using ADEPT algorithm. From these results, a twenty-marker-panel embodiment of the invention was selected using the projection pursuit technique including Prolactin, TTR, CA 19-9, ULBP-2, CD40L, MMP-9, FSH, Adiponectin, TSH, ACTH, Eotaxin, TNFR-I, EGFR, IL-8, S-100, AFP, Mesothelin, MMP-3, CEA, IGFBP-I. The resulting model led to correctly classifying 99% of the test set observations, with a sensitivity of 99% at a specificity of 99%. These results indicate that combining multiple biomarkers results in higher diagnostic power for endometrial cancer than that offered by each individual biomarker. Applying such a multimarker panel does not result in overfitting since the same panel demonstrated similar but 3-5% lower classification when logistic regression or classification trees algorithms were used.

These results demonstrate that analysis of multiple serum biomarkers using the multiplexing xMAP® technology allowed identification of marker panels with the high sensitivity and specificity for discrimination of endometrial cancer vs. healthy controls in a cross-validation case-control set.

The invention claimed is:

1. A method of diagnosing endometrial cancer comprising: determining the levels of at least two markers in the blood of a patient selected from the group consisting of CA 125, CA 15-3, CA 19-9, CEA, AFP, CA 72-4, VEGF, bFGF, IGFBPI, HGF, ErbB2, EGFR, TGFα, Fas, FasL, Cyfra 21-1, MMP-1, MMP-2, MMP-3, MMP-7, MMP-8, MMP-9, MMP-12, MMP-13, tPAI, sICAM, sVCAM, sE-selectin, adiponectin, resistin, IL-6, IL-8, TNFα, TNFR I, G-CSF, CD40L, IL-2R, IP-10, MCP-1, MIP-1α, MIP-1β, MIF, eotaxin, RANTES, FSH, LH, TSH, ACTH, Prolactin, GH, βHCG, hK8, hK10, active PAI-1, ULBP-1, ULBP-2, ULBP-3, MICA, angiostatin, SCC, serum amyloid A, TTR, S100, mesothelin, and myeloperoxidase (MPO), wherein the dysregulation of said at least two markers indicates high specificity and sensitivity for a diagnosis of endometrial cancer.

2. The method according to claim 1 wherein at least five markers in the blood of a patient selected from the group consisting of CA 125, CA 15-3, CA 19-9, CEA, AFP, CA 72-4, VEGF, bFGF, IGFBPI, HGF, ErbB2, EGFR, TGFα, Fas, FasL, Cyfra 21-1, MMP-1, MMP-2, MMP-3, MMP-7, MMP-8, MMP-9, MMP-12, MMP-13, tPAI, sICAM, sVCAM, sE-selectin, adiponectin, resistin, IL-6, IL-8, TNFα, TNFR I, G-CSF, CD40L, IL-2R, IP-10, MCP-1, MIP-1α, MIP-1β, MIF, eotaxin, RANTES, FSH, LH, TSH, ACTH, Prolactin, GH, βHCG, hK8, hK10, active PAI-1, ULBP-1, ULBP-2, ULBP-3, MICA, angiostatin, SCC, serum amyloid A, TTR, S100, mesothelin, and myeloperoxidase (MPO), wherein the dysregulation of said at least five markers indicates high specificity and sensitivity for a diagnosis of endometrial cancer.

3. The method according to claim 1 wherein at least eight markers in the blood of a patient selected from the group consisting of CA 125, CA 15-3, CA 19-9, CEA, AFP, CA 72-4, VEGF, bFGF, IGFBPI, HGF, ErbB2, EGFR, TGFα, Fas, FasL, Cyfra 21-1, MMP-1, MMP-2, MMP-3, MMP-7, MMP-8, MMP-9, MMP-12, MMP-13, tPAI, sICAM, sVCAM, sE-selectin, adiponectin, resistin, IL-6, IL-8, TNFα, TNFR I, G-CSF, CD40L, IL-2R, IP-10, MCP-1, MIP-1α, MIP-1β, MIF, eotaxin, RANTES, FSH, LH, TSH, ACTH, Prolactin, GH, βHCG, hK8, hK10, active PAI-1, ULBP-1, ULBP-2, ULBP-3, MICA, angiostatin, SCC, serum amyloid A, TTR, S100, mesothelin, and myeloperoxidase (MPO), wherein the dysregulation of said at least eight markers indicates high specificity and sensitivity for a diagnosis of endometrial cancer.

4. The method according to claim 1 wherein one marker comprises Prolactin.

5. A method of diagnosing endometrial cancer comprising determining the levels of prolactin, GH, eotaxin, e-selectin and FSH wherein the dysregulation of prolactin, GH, eotaxin, e-selectin and FSH indicates high specificity and sensitivity for a diagnosis of endometrial cancer.
